(19) United States
(12) Patent Application Publication
(10) Pub. No.: US 2026/0036539 A1
(43) Pub. Date: Feb. 5, 2026

(54) DEVICE AND METHOD FOR MONITORING AND REAL-TIME DETECTING AGENTS OF INFECTIONS FROM CLINICAL SPECIMENS

(71) Applicant: Tao Treasures LLC DBA Nanobiofab, Frederick, MD (US)

(72) Inventors: Xiaonao LIU, Frederick, MD (US); Derese GETNET, Baltimore, MD (US); Taejun KO, Clarksburg, MD (US); Robert HOPKINS, Frederick, MD (US); Deyu LIU, Richland, WA (US); Buyu YEH, Frederick, MD (US); Jennifer DOOTZ, Boonsboro, MD (US)

(21) Appl. No.: 19/289,844

(22) Filed: Aug. 4, 2025

Related U.S. Application Data
(60) Provisional application No. 63/678,801, filed on Aug. 2, 2024.
(60) Provisional application No. 63/792,083, filed on Apr. 21, 2025.

(51) Int. Cl. G01N 27/12; A61M 1/00; A61M 27/00

(52) U.S. Cl. CPC G01N 27/12; A61M 1/95; A61M 27/00; A61M 2205/3303

(57) ABSTRACT

A device and method are disclosed for the real-time monitoring and detection of infectious agents in clinical specimens. The device utilizes a sensor array to detect gaseous biosignatures, such as volatile organic compounds (VOCs), released by metabolically active pathogens. A data processing module with an artificial intelligence (AI) algorithm analyzes multi-sensor data to generate a time-resolved biosignature profile. The algorithm interprets this profile to provide diagnostic outputs, including pathogen presence, identity, an estimation of microbial load (e.g., CFU/mL), and, notably, a determination of the microbial growth rate calculated from the profile's change over time. Embodiments of the device include handheld point-of-care analyzers, integrated ‘smart caps’ for specimen containers, and in-line monitors for surgical drains. The technology enables rapid, data-driven clinical decisions for infection management by providing timely and dynamic assessments of microbial activity.

### CROSS-REFERENCE TO RELATED APPLICATIONS

This application claims the benefit of priority under 35 U.S.C. § 119 to U.S. Provisional Application No. 63/678,801, filed Aug. 2, 2024 and U.S. Provisional Application No. 63/792,083, filed Apr. 21, 2025, the entire contents of which are incorporated herein by reference in their entireties.

### TECHNICAL FIELD

The present invention generally relates to a device for monitoring and real-time detecting agents of infections from clinical specimens. More specifically, it relates to an integrated medical device that detects pathogen presence, identifies pathogen type, monitors microbial growth, or thus predicates infections in a surgical procedure.

### BACKGROUND

Orthopedic surgery encompasses a broad range of procedures aimed at diagnosing and treating conditions affecting the musculoskeletal system, including bones, joints, ligaments, tendons, and muscles. Common orthopedic surgeries include joint replacements, spine surgery, hand and wrist surgery, foot and ankle surgery, and various sports medicine procedures. These surgeries, while often necessary for restoring function and reducing pain, carry risks of post-surgical complications, with infection being one of the most serious concerns.

In particular, Prosthetic Joint Infections (PJIs) represent a significant complication in joint replacement surgeries, often leading to severe consequences such as joint destruction, prolonged hospital stays, and the need for revision surgeries or even amputation. Similarly, other orthopedic surgeries, such as spine or foot and ankle surgeries, are also susceptible to post-operative infections, which can drastically affect patient recovery and outcomes.

The post-surgical infections also presents a high risk to breast surgery, including reconstructive procedures and surgeries involving implants. These infections, often occurring at the surgical site or around implants, can lead to serious complications, requiring additional treatments or interventions and prolonging the patient's recovery time.

Currently, the diagnosis of infections in both orthopedic and breast surgeries relies on a combination of clinical evaluations, microbiological testing, and imaging techniques. These methods, while effective, often face limitations such as delayed results, low specificity, and the invasive nature of sample collection, creating a critical gap in timely and accurate post-surgical infection management. For example, traditional methods of detecting infections in joint or synovial fluid involve cell counts, gram staining, and microbial cultures, which may fail to identify early-stage or low-grade infections.

In the case of breast surgery, particularly those involving implants, diagnosing infections can be challenging due to the limitations of current diagnostic methods, which often require invasive procedures and time-consuming culture processes. Additionally, the emergence of multi-drug resistant organisms further complicates the management and treatment of post-surgical infections.

There is a pressing need for a device that can provide real-time, accurate, and non-invasive assessment of microbial status in clinical specimens associated with a specific surgical procedure. The analysis of gaseous biosignatures, particularly, volatile organic compounds (VOCs), in bodily fluids offers a promising alternative for the rapid and non-invasive diagnosis of infections. VOCs are metabolic by-products released by bacteria and fungi that can be detected in fluids such as synovial fluid and post-surgical drain fluid. By profiling these VOCs, it is possible to detect the presence of an infection and identify the specific pathogens involved.

This technology allows for near-immediate, direct, and potentially continuous monitoring of indicators associated with infection or microbial proliferation by analyzing the gaseous profile, potentially eliminating the need for otherwise time-consuming monitoring methods, reducing the risks of post-surgical infections, ultimately simplifying operational processes in hospital surgical rooms while improving patient outcomes.

### SUMMARY OF THE INVENTION

The present disclosure is directed to solving the technical problems in the prior art related to the time-consuming, complex, and static nature of detecting infections in clinical specimens. The present invention provides a device, system, and method for the real-time detection, dynamic monitoring, identification, and quantification of pathogenic agents in clinical specimens, such as body fluids (synovial fluid, drain fluid, urine, blood, cerebrospinal fluid (CSF), saliva, bronchial lavages, wound exudates) and tissues. The invention achieves rapid, minimally invasive, or non-invasive infection diagnosis and monitoring by analyzing gaseous biosignatures, including, but not limited to, volatile organic compounds (VOCs), sulfur-containing compounds, nitrogen-containing compounds, and other metabolic gases, released by metabolically active pathogens.

Accordingly, one aspect of the present invention provides a device for analyzing a clinical specimen to detect, monitor, and/or quantify pathogens therein. The device typically comprises:
a) A sensor array configured to interface with a clinical specimen. The sensor array comprises a plurality of sensors, wherein: at least one chemiresistive gas sensor positioned to detect gaseous analytes released from the clinical specimen.

In some embodiments, to enhance detection accuracy and specificity through multi-modal data fusion, the sensor array further comprises one or more auxiliary sensors. Said auxiliary sensors are selected from the group consisting of: a pH sensor, a temperature sensor, a humidity sensor, an optical sensor, and an impedance/conductance sensor, which are positioned to measure properties of the specimen through direct contact or proximity.
b) A data processing module communicatively coupled to said sensor array. The module is configured to execute operations comprising:
i. Receiving a plurality of sensor signals from the sensor array over either a short-duration (spot-check) cycle or a prolonged period (time-resolved monitoring) to generate biosignature profile.
ii. Processing said biosignature profile using one or more pre-trained machine learning models to generate at least one of the following diagnostic outputs:
   (1) An indication of pathogen presence, identifying the specimen as positive or negative for infection based on the profile's deviation from a baseline or negative control.
   (2) An assessment of pathogen identity, aiding in the classification of the pathogen (e.g., as Gram-positive, Gram-negative) or the identification of a specific species by pattern-matching the profile against a library of known pathogen signatures.
   (3) An estimation of microbial load, correlating key features of the biosignature profile (e.g., peak signal intensity, area-under-the-curve) with a predicted pathogen concentration, expressed in Colony Forming Units per milliliter (CFU/mL) or per gram (CFU/g), based on a pre-established calibration model.
   (4) A determination of microbial growth rate, calculated from a rate of change (i.e., the first derivative) of said biosignature profile over time. A persistent positive rate of change is indicative of active microbial proliferation, enabling predictive monitoring of infection development.
c) A user interface for displaying, transmitting, or providing an alert based on said diagnostic output. The interface may comprise an integrated display screen (e.g., LCD), status indicators (e.g., LEDs), an audible alarm, and/or a wireless communication module (e.g., Bluetooth, Wi-Fi) for transmitting data to an external device, such as a smartphone, tablet, or hospital information system.

The sensor array comprises electrochemical sensors, infrared sensors, photoionization detectors, humidity sensors, thermal sensors, pH sensors, viscosity sensors, and chemiresistive sensors made from materials including conductive polymers, carbon nanotubes, graphene, transition metal chalcogenides, metal nitrides, metal sulfides, MXenes, metal-organic frameworks (MOFs), composite nanomaterials, and various metal oxides.

In preferred embodiments, the device of the present invention is configured in various forms to suit different clinical workflows. For example, the device may be a standalone, portable unit for “spot-check” analysis of collected samples. In another embodiment, the device is integrated into a “smart cap” for a sample collection container (e.g., a urine cup or fluid aspirate tube). In yet another embodiment, the device is designed as an in-line monitoring module for integration with existing medical equipment, such as a dressing, surgical drainage system or a Negative Pressure Wound Therapy (NPWT) device.

Another aspect of the present invention provides a method for monitoring a pathogenic infection in a clinical specimen. The method comprises the steps of:
a) Exposing a sensor array of a monitoring device to a headspace gas emanating from the clinical specimen.
b) Acquiring, via a data processing module, a plurality of sensor signals from the sensor array over a period of time to generate a time-resolved biosignature profile.
c) Analyzing said time-resolved biosignature profile with a pre-trained computational model to determine at least one characteristic of the specimen, said characteristic being selected from the group consisting of: pathogen presence, pathogen identity, microbial load, and microbial growth rate.
d) Reporting the at least one determined characteristic to a user via a user interface. This step may be performed in real-time or upon completion of the analysis.

Through the foregoing device and method, the present invention provides immediate feedback and continuous dynamic data on infection status without the need for complex sample preparation or lengthy culture periods. This not only reduces the time to diagnosis but also enables clinicians to track the evolution of an infection in real-time (e.g., proliferation, stabilization, or resolution following therapy), thereby facilitating more timely and precise clinical decision-making and ultimately improving patient outcomes.

### BRIEF DESCRIPTION OF THE DRAWINGS

FIG. 1 illustrates the system architecture, including the sensor array, data processing module, and user interface, highlighting data flow from specimen exposure to diagnostic output. Specifically, the diagram begins with the clinical specimen source, such as synovial fluid, drain fluid, or tissue. Gaseous biosignatures (e.g., volatile organic compounds [VOCs], hydrogen sulfide [H₂S]) emitted from the clinical specimen are exposed to the sensor array (200), comprising chemiresistive gas sensors and auxiliary sensors (e.g., pH, temperature, optical sensors). Raw sensor signals collected by the sensor array are transmitted to the data processing module (300), where the signals undergo signal processing, feature extraction, and analysis via artificial intelligence/machine learning (AI/ML) models. The data processing module outputs diagnostic results to the user interface and reporting module (400), providing clear, actionable information to the user through displays, alerts, data logging, and wireless transmission to external electronic health record (EHR) systems or mobile applications.

FIG. 2 details the sensor array's multi-modal design, featuring chemiresistive and auxiliary sensors and nanoporous sensing materials interaction with gas molecules. Specifically, the primary view depicts a top-down layout of a microchip hosting multiple sensing elements, including chemiresistive sensors and auxiliary sensors (e.g., pH and temperature sensors). An enlarged inset provides a detailed cross-sectional representation of a typical chemiresistive sensor, clearly showing the layered structure consisting of a substrate base, metallic interdigitated electrodes, and a top porous layer composed of nanoporous sensing material. Gas molecules, such as volatile organic compounds (VOCs), are illustrated adsorbing onto the sensor surface, demonstrating the principle behind sensor detection and signal generation. This multi-modal design enables simultaneous acquisition of diverse biosignature data, significantly enhancing the accuracy and specificity of pathogen detection and identification in clinical specimens.

FIG. 3 demonstrates sensor response curves under normal and infection conditions, illustrating key diagnostic signal features. Specifically, the response curves generated by the sensor array when exposed to clinical specimens under two distinct scenarios: a negative (normal or sterile) condition (left panel), and a positive (infection present) condition (right panel). The curves clearly highlight differential sensor behavior and measurable signals that arise in the presence of infection-related volatile organic compounds (VOCs). Notable features captured include rapid signal rise, response peaks, and plateau phases, all of which are key characteristics utilized for feature extraction and diagnostic interpretation by the data processing module. These response curves enable robust differentiation between infected and non-infected specimens, providing critical, actionable information for clinical decision-making.

FIG. 4 shows a handheld analyzer embodiment with external ergonomic design and internal cross-sectional layout, including analysis chamber, sensor array, data module, and battery. Panel (A) provides an external 3D perspective view of the handheld device, highlighting its ergonomic form factor designed for easy handling during clinical procedures. The device includes a digital touchscreen display for result visualization and user interaction, control buttons beneath the screen for basic device operations, and a prominently positioned sample inlet employing a standardized Luer lock connector for secure fluid sample injection.

Part (B) depicts an internal cross-sectional view of the same handheld analyzer, revealing the layout of the critical components within the device housing. The illustration clearly shows the analysis chamber, where the clinical specimen is introduced and exposed to the sensor array (200). The sensor array is positioned immediately adjacent to the analysis chamber for effective detection and measurement of gaseous biosignatures. Beneath the sensor array is the data processing module (300), mounted on a printed circuit board (PCB), responsible for signal processing, data analysis, and diagnostic output generation. A rechargeable battery at the bottom of the device provides portable power, ensuring suitability for point-of-care and field use. Arrows and component placement indicate the logical flow and interactions between these internal parts.

FIG. 5 depicts the Smart Cap embodiment, illustrating practical usage and internal dual-compartment design separated by a sterile, gas-permeable membrane. Panel (A) depicts the practical usage scenario, showcasing a typical transparent specimen vial containing a clinical fluid (e.g., urine, fluid aspirate), sealed securely by the disc-shaped Smart Cap (Embodiment 2).

Part (B) provides an enlarged, detailed cross-sectional view of the Smart Cap. This cross-section clearly illustrates the two-compartment internal design. The upper, sealed compartment houses essential electronic components, including a coin-cell battery and a wireless communication module mounted on a printed circuit board (PCB). The lower compartment is the sensing chamber, containing the miniaturized sensor array (200), strategically positioned to detect gaseous biosignatures emanating from the specimen headspace. Critically, the two compartments are separated from direct specimen exposure by a sterile, gas-permeable membrane, ensuring sensor protection, sample integrity, and sterility. The Smart Cap design emphasizes ease-of-use, sterility maintenance, and wireless connectivity, significantly enhancing clinical specimen handling and analysis efficiency.

FIG. 6 illustrates an in-line monitor embodiment integrated into surgical drainage tubing, detailing the application scenario. Panel (A) Application Scenario: This view depicts the typical clinical installation, clearly demonstrating how the in-line monitor is integrated directly within a surgical drain tubing. The module is shown positioned between the patient surgical site and a fluid collection drainage bag, allowing continuous monitoring of gaseous biosignatures released from the fluid flowing through the tubing.

Part (B) Cross-Sectional Structure: This detailed cross-sectional illustration reveals the internal in-line structural design of the module. It clearly distinguishes the main fluid flow channel from the adjacent sensing chamber. A sterile, gas-permeable membrane separates the sensing chamber from direct fluid contact, permitting gases emitted from the fluid to diffuse into the sensing chamber and interact with the sensor array without contaminating the sensors. This innovative structure enables continuous and stable monitoring, minimizes sensor fouling, and preserves fluid sterility, providing reliable early detection and dynamic tracking of post-surgical infections.

FIG. 7 presents a method-of-use flowchart detailing operational steps from initialization, specimen introduction, measurement initiation, AI/ML analysis, to result reporting and data logging. The illustrated workflow includes the sequential steps of: (1) device initialization and patient/sample association, including powering on the device, performing self-check routines, and linking the device to a specific patient or sample; (2) clinical specimen introduction, demonstrating different specimen handling according to specific embodiments (e.g., handheld analyzer, smart cap, or in-line monitor); (3) initiation of measurement and data acquisition, including equilibration and measurement cycles (spot-check or continuous modes); (4) data processing and analysis using artificial intelligence/machine learning (AI/ML) algorithms, encompassing signal pre-processing, feature extraction, and diagnostic interpretation; (5) displaying diagnostic results clearly and immediately, providing actionable information for clinical decision-making; and (6) logging the data internally and wirelessly transmitting results to external electronic health record systems or other clinical management platforms. This structured workflow diagram explicitly supports the practical implementation of the invention's method claims, ensuring streamlined integration into existing clinical practices.

FIG. 8 provides representative user interfaces for diagnostic results and continuous monitoring outputs, demonstrating predictive microbial growth tracking. Panel (A) shows a representative diagnostic result screen from a handheld device or mobile application. The interface clearly indicates pathogen detection results (e.g., “Positive: Pseudomonas aeruginosa detected”), along with a microbial load assessment (“Microbial Load: HIGH”), timestamp, patient identification, and intuitive visual status indicators.

Part (B) presents a graphical interface displaying continuous monitoring data, with microbial load (signal intensity) plotted over time (hours). The rising trend line clearly demonstrates increasing microbial growth rate, providing clinicians with predictive insights into infection progression. Key alert thresholds and triggers are visibly indicated on the graph, underscoring the system's clinical utility in proactive patient care management.

FIG. 9 visually illustrates clinical use cases: Panel (A) Synovial Fluid Aspiration depicts rapid diagnosis of prosthetic joint infections (PJI). Synovial fluid is aspirated from a patient's knee joint and immediately introduced into a specimen vial sealed with the integrated Smart Cap embodiment of the invention, enabling real-time pathogen detection and analysis.

Part (B) Continuous Monitoring of a Surgical Site Infection (SSI) shows the in-line monitoring embodiment integrated within a surgical drainage system. The monitoring device is placed between the surgical incision site and the drainage bag, allowing continuous real-time detection of microbial growth indicators in postoperative fluid, facilitating early warning and prompt intervention for potential infections.
Part (C) Spot-Check Assessment of Chronic Wounds illustrates direct, non-invasive spot-check assessment of a chronic wound, such as a diabetic foot ulcer, using the handheld analyzer embodiment. The device directly analyzes gaseous biosignatures emitted from the wound without physical contact, providing immediate feedback on microbial presence and load, thus guiding timely clinical decision-making and wound management.

### DETAILED DESCRIPTION

The following detailed description, in conjunction with the accompanying drawings, provides a more complete understanding of the disclosure and its various embodiments. The description is not intended to be limiting, and modifications and variations within the scope of the disclosure will be apparent to those skilled in the art. The invention is capable of other embodiments and of being practiced and carried out in various ways.

This application incorporates by reference the entire contents of U.S. Provisional Application No. 63/678,801, filed Aug. 2, 2024 and U.S. Provisional Application No. 63/792,083, filed Apr. 21, 2025. Further, the embodiments of the device and method include, but are not limited to, the embodiments as disclosed, described, and/or referred to in U.S. application Ser. No. 18/829,748, filed Sep. 10, 2024; and U.S. application Ser. No. 18/933,674, filed Oct. 31, 2024, which are all hereby incorporated by reference in their entireties. The embodiments in these applications herein incorporated can be regarded in combination with one another or as a single invention, rather than as discrete and independent filings.

The present invention is directed to a device and method for the real-time monitoring and detection of infectious agents from clinical specimens. The core of the invention lies in its ability to analyze gaseous biosignatures released by metabolically active pathogens. This allows for a rapid, non-invasive or minimally invasive assessment of a specimen's microbial status, overcoming the delays and limitations of traditional culture-based methods. The interpretation of these biosignatures aims to generate clinically actionable outputs, including but not limited to: an indication of pathogen presence; an estimation of microbial load; an assessment aiding in pathogen identification; differentiation between infectious and non-infectious inflammatory states; and, crucially, an estimation of microbial growth rate based on the temporal evolution of the biosignature profile.

### I. System Architecture and Overall Operation

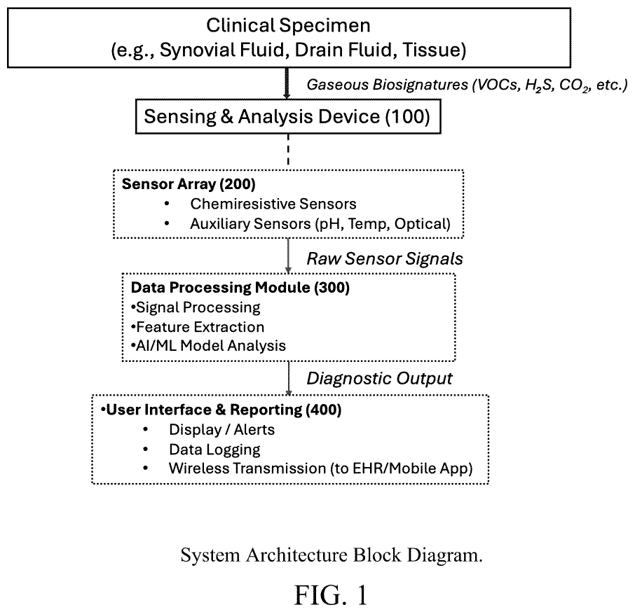

Referring now to FIG. 1, a block diagram illustrates an exemplary apparatus system 100 according to one embodiment of the disclosure. The system 100 is specifically designed for the real-time detection, identification, and continuous monitoring of pathogens in clinical specimens by analyzing gaseous biosignatures (such as volatile organic compounds [VOCs], hydrogen sulfide [H₂S], and others) emitted from the specimens. In operation, the system begins with the exposure of a sensor array 200 to the gaseous analytes released from a clinical specimen (such as synovial fluid, drainage fluid, tissue samples, or wound exudate). This sensor array 200 comprises multiple sensing elements, including primary chemiresistive sensors designed for direct detection of gaseous biosignatures, and auxiliary sensors (e.g., pH, temperature, optical sensors) enhancing the detection accuracy and specificity through multi-modal data fusion. Interaction between gaseous analytes and sensors within the sensor array 200 generates distinct physical and electrical changes (such as changes in resistance, impedance, optical properties, or other measurable parameters). These changes constitute raw sensor signals, which are then transmitted to the data processing module 300. The data processing module 300, operatively connected to the sensor array 200, executes several critical steps including initial signal processing (filtering, noise reduction, amplification), feature extraction from raw signals, and subsequent diagnostic analysis using advanced artificial intelligence/machine learning (AI/ML) models. This module generates a biosignature profile uniquely indicative of microbial presence, identity, microbial load, and growth trends. Finally, the processed and interpreted diagnostic results from the data processing module 300 are transmitted to the user interface and reporting module 400. This module provides clear, actionable information directly to clinical users via visual displays, audible or visual alerts, secure data logging, and wireless transmission capabilities to external systems such as electronic health records (EHR) or mobile healthcare applications.

The described detection and monitoring method can operate continuously to provide uninterrupted real-time monitoring of microbial changes, or periodically in a “spot-check” mode, allowing flexibility in clinical decision-making across various healthcare scenarios.

### II. The Sensor Array (200): Principles and Composition

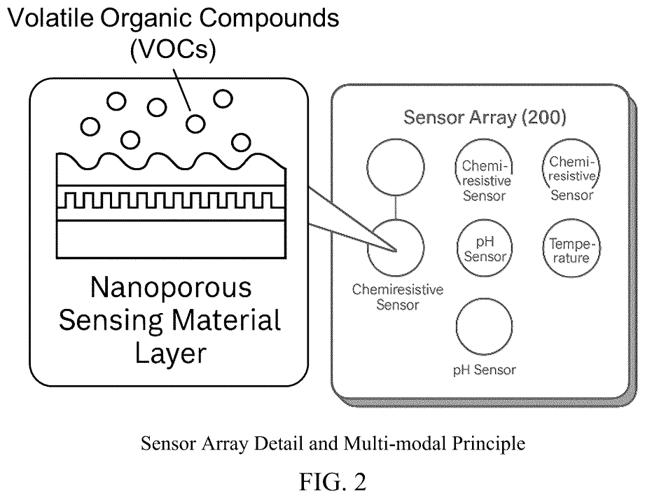

Referring now to FIG. 2, the sensor array 200 serves as the primary sensing transducer of the system, generating a rich, multi-dimensional dataset by detecting gaseous emissions from clinical specimens. The array is not a single sensor but a collection of diverse sensing elements, each providing a unique response to the complex mixture of analytes. The array may comprise a plurality of sensors, for example, between 2 and 32 sensors or more, allowing for the creation of a highly specific “fingerprint” for different microbial states.

### A. Core Sensing Elements: Chemiresistive Sensors

Chemiresistive sensors operate by detecting changes in electrical resistance upon adsorption or interaction of gas molecules with a nanoporous sensing material layer. As clearly depicted in the enlarged cross-sectional inset of FIG. 2, each chemiresistive sensor typically includes a base substrate layer supporting metallic interdigitated electrodes, covered by a nanoporous sensing material specifically engineered for gas analyte interaction.

Target Analytes: The sensor array is configured to respond to one or more compounds in the gas phase, including but not limited to:
1. Volatile Organic Compounds (VOCs): Such as short-chain fatty acids (e.g., acetic acid, propionic acid, butyric acid, isovaleric acid), alcohols (e.g., ethanol, isopropanol), aldehydes, ketones (e.g., acetone), esters, amines, and terpenes.
2. Sulfur-Containing Compounds: Such as hydrogen sulfide, dimethyl sulfide, dimethyl disulfide, and methanethiol, which are often characteristic of anaerobic bacterial metabolism.
3. Nitrogen-Containing Compounds: Such as ammonia, trimethylamine, indole, and skatole, which are common byproducts of amino acid and protein degradation.
4. Other Indicator Gases: Such as carbon dioxide (CO₂), the concentration of which can indicate the overall rate of metabolic activity.

The sensing materials employed in the sensor array 200 depicted in FIG. 2 may comprise one or more elements, or compounds thereof (e.g., oxides, sulfides, nitrides), as well as conductive polymers and composite materials. Suitable elements that may form part of the sensing material include, but are not limited to: Sn, Co, Zn, In, Cu, Ni, Cr, Mn, W, Ti, V, Fe, Al, Ga, Ag, Au, Pd, Pt, Si, Ce, Mo, Zr, La, Y, Mg, Nb, Ru, and Te, and others, allowing tunable selectivity and sensitivity for target gases.

These elements are combined to form compounds with highly tunable sensing properties. In particular, the metal oxides can be composed of unary, binary, ternary, quaternary, quinary, senary, septenary, and even octonary multiple-component metal oxides, allowing for the engineering of highly complex and selective sensing surfaces. Illustrative examples of suitable sensing materials include, but are not limited to:
a) Binary Metal Oxides: Pure or mixed phases of binary composition, such as Tin Oxide (SnO₂), Zinc Oxide (ZnO), Tungsten Oxide (WO₃), Titanium Dioxide (TiO₂), Copper Oxide (CuO, Cu₂O), Nickel Oxide (NiO), Iron Oxides (Fe₂O₃, Fe₃O₄), Indium Oxide (In₂O₃), Cerium Oxide (CeO₂), Zirconium Oxide (e.g., ZrO₂), Manganese Oxide (e.g., MnO₂, Mn₃O₄), Cobalt Oxide (e.g., CoO, Co₃O₄), Gallium Oxide (e.g., Ga₂O₃), Molybdenum Oxide (e.g., MoO₃), Magnesium Oxide (e.g., MgO), or Niobium Oxide (e.g., Nb₂O₅).
b) Mixed or Ternary Metal Oxides: Pure ternary crystal phases or doped binary phases, such as Zinc Stannate (Zn₂SnO₄), Indium Tin Oxide (ITO), perovskite oxides (e.g., SrTiO₃, LaCoO₃), or spinel oxides (e.g., NiFe₂O₄, NiCo₂O₄).
c) Metal Sulfides or Nitrides: Such as Molybdenum Disulfide (MoS₂), Tungsten Disulfide (WS₂), Tin Sulfide (SnS, SnS₂), and Gallium Nitride (GaN).
d) Noble Metal Catalysts/Additives: Noble metals like Au, Pt, Pd, Ag may be included as dopants or as nanoparticles decorating the surface of the primary sensing material (e.g., Pd-doped SnO₂, Au nanoparticles on ZnO) to enhance sensitivity and selectivity.
e) Carbon-Based Materials: Graphene, graphene oxide (GO), reduced graphene oxide (rGO), and carbon nanotubes (CNTs), which may be used alone or composited with other materials.
f) Conductive Polymers: Such as polyaniline (PANI), polypyrrole (PPy), and PEDOT:PSS.
g) Combinations and Heterostructures: Direct mixtures, composites (e.g., polymer-metal oxide composites), or heterostructures (e.g., p-n junctions like CuO/ZnO) comprising two or more of the above material types.

Sensor Morphology: The physical structure of the sensing material is also critical. The material may comprise one or more nanoporous structures, such as macroporous, mesoporous, microporous, or hierarchical porous structures (e.g., combining micropores and mesopores), to maximize the surface area available for gas interaction, thereby enhancing sensitivity.

### B. Auxiliary Sensing Elements (for Multi-Modal Fusion)

In preferred embodiments, the sensor array 200 is a multi-modal system, integrating auxiliary sensors to gather orthogonal data streams for a more robust and accurate diagnosis. These optional sensors may include:
1. Electrochemical Sensors: These sensors can measure a wide range of markers through redox reactions. They may be amperometric (measuring current at a set potential, e.g., for hydrogen peroxide), potentiometric (measuring potential, e.g., using ion-selective electrodes for pH or specific ions like K+, Na+, Cl−), conductometric/impedimetric (measuring changes in electrical conductivity/impedance of a fluid related to ionic strength or cell lysis), or voltametric (measuring current as potential is varied to characterize redox species).
2. pH Sensors: Pathogenic metabolism frequently alters the local pH of body fluids or tissues. For example, fermentation produces acids, while urease activity produces ammonia, raising the pH. A pH sensor provides a direct measurement of this critical biochemical change.
3. Thermal Sensors: High-precision thermal sensors can detect temperature variations. Such changes may be indicative of the exothermic nature of rapid microbial metabolism or the host's localized inflammatory response, providing another key indicator of infection. It also provides necessary calibration parameters for the algorithm.
4. Optical Sensors: Operating in the visible and/or near-infrared range, optical sensors can measure changes in the physical properties of the analytical substrate. They can detect increased turbidity or optical density caused by bacterial proliferation, or specific color changes associated with pigments produced by certain pathogens (e.g., pyocyanin from P. aeruginosa).
5. Humidity Sensors: A humidity sensor monitors the moisture content within the sample environment. This data is crucial for assessing the state of a wound and, importantly, for calibrating and normalizing the signals from the chemiresistive gas sensors, whose performance can be humidity-dependent.

### III. Typical Sensor Response Curves Under Normal and Infection Conditions

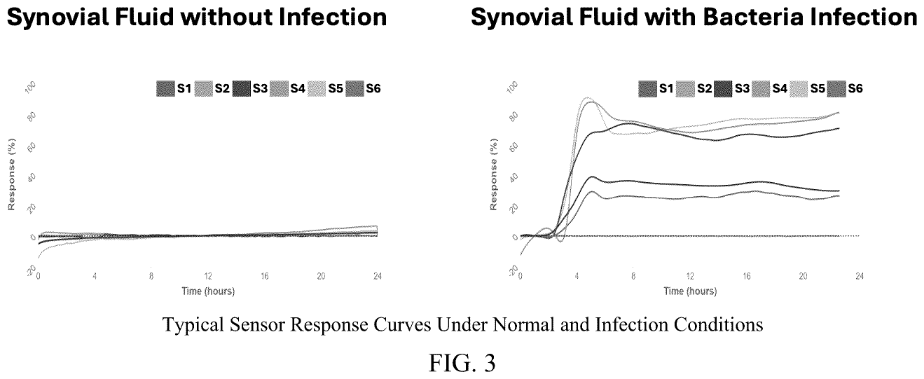

Referring now specifically to FIG. 3, exemplary response curves generated by the sensor array when exposed to clinical specimens are shown under two distinct conditions: negative (normal or sterile) and positive (infection present). In the negative condition (left panel), sensor responses remain at baseline or show minimal fluctuations, indicative of the absence of microbial metabolic activity and low VOC concentrations. Conversely, the positive condition (right panel) demonstrates distinctive and rapid increases in sensor signals, clearly reflecting active microbial metabolism and elevated concentrations of infection-associated VOCs and other gaseous analytes.

Notable diagnostic features observable from these curves include rapid signal rise, distinct response peaks, stabilization plateau phases, and sustained elevated signal intensities. These measurable characteristics enable the data processing module (300) to execute precise feature extraction (peak height, area-under-the-curve, rate of signal change), facilitating robust differentiation between infected and non-infected states. Thus, these characteristic response curves form a critical basis for real-time, reliable diagnostic interpretations, allowing clinicians to rapidly and confidently make informed clinical decisions.

### III. Target Clinical Specimens and Pathogens

Applicable Clinical Specimens: The device and method of the present invention are designed for versatility and can be applied to a wide range of clinical specimens. These include, but are not limited to, skin or wound bed, body fluids such as blood, synovial fluid, surgical drain fluid, urine, cerebrospinal fluid (CSF), saliva, bronchial lavages, pus, serous fluid, serosanguineous fluid, lymph, bile, and wound exudate. The invention is also applicable to the analysis of tissue samples, such as biopsies, where gaseous emissions can be analyzed from the tissue surface.

Target Pathogenic Microorganisms: The system is capable of detecting a comprehensive range of pathogens known to cause infections. The pathogens can be one or more bacterial or fungal species, such as Acinetobacter anitratus, Acinetobacter baumannii, Actinomyces israelii, Agrobacterium radiobacter, Agrobacterium tumefaciens, Anaplasma phagocytophilum, Aspergillus fumigatus, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Candida albicans, Helicobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Candida species, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus gallinarum, Enterococcus maloratus, Escherichia coli, Francisella tularensis, Fusobacterium nucleatum, Enterobacter species, Fusarium solani, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactobacillus delbrueckii, Lactococcus lactis, Legionella pneumophila, Listeria monocytogenes, Methylobacterium extorquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexicoense, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faecium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to include one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae. The fungal species include but not limited to Aspergillus Species, Candida Species, Fusarium Species, Mucorales (Zygomycetes), Scedosporium Species, Curvularia Species, Alternaria Species, Trichophyton Species, Exophiala Species, Cladosporium Species, Bipolaris Species, Penicillium Species, Phialophora and Fonsecaea Species (Agents of Chromoblastomycosis). The pathogens further include, but are not limited to: Cutibacterium acnes (formerly Propionibacterium acnes), other coagulase-negative Staphylococcus species (e.g., S. lugdunensis, S. capitis, S. warneri, S. hominis), Ureaplasma species (e.g., U. parvum), Nocardia species (e.g., N. asteroides, N. farcinica), anaerobic Gram-positive cocci such as Finegoldia magna and Parvimonas micra, Tropheryma whipplei, and zoonotic bacteria including Capnocytophaga canimorsus. Fungal pathogens may additionally include Candida auris, Cryptococcus neoformans, and Coccidioides species, among others.

The Antibiotic-resistant bacterial strains may include Carbapenem-resistant Acinetobacter baumannii, carbapenem-resistant Pseudomonas aeruginosa, vancomycin-resistant Enterococcus faecium, methicillin-resistant Staphylococcus aureus, vancomycin-resistant Staphylococcus aureus, clarithromycin-resistant Helicobacter pylori, fluoroquinolone-resistant Campylobacter coli, fluoroquinolone-resistant Campylobacter fetus, fluoroquinolone-resistant Campylobacter jejuni, fluoroquinolone-resistant Helicobacter pylori, fluoroquinolone-resistant Salmonella enteritidis, fluoroquinolone-resistant Salmonella typhi, fluoroquinolone-resistant Salmonella typhimurium, cephalosporin-resistant Neisseria gonorrhoeae, fluoroquinolone-resistant Neisseria gonorrhoeae, penicillin-non-susceptible Streptococcus pneumoniae, ampicillin-resistant Haemophilus influenzae, fluoroquinolone-resistant Shigella dysenteriae, carbapenem-resistant Escherichia coli, carbapenem-resistant Klebsiella pneumoniae, carbapenem-resistant Enterobacter cloacae, carbapenem-resistant Serratia marcescens, carbapenem-resistant Proteus vulgaris, carbapenem-resistant Proteus mirabilis, carbapenem-resistant Proteus penneri, carbapenem-resistant Providencia stuartii, carbapenem-resistant Morganella morganii, cephalosporin-resistant Escherichia coli, cephalosporin-resistant Klebsiella pneumoniae, cephalosporin-resistant Enterobacter cloacae, cephalosporin-resistant Serratia marcescens, cephalosporin-resistant Proteus vulgaris, cephalosporin-resistant Proteus mirabilis, cephalosporin-resistant Proteus penneri, cephalosporin-resistant Providencia stuartii and cephalosporin-resistant Morganella morganii.

### IV. Data Processing Module (300) and Analytical Methodology

The data processing module 300 serves as the computational engine of the device. It comprises hardware components such as a micro-controller unit (MCU), a digital signal processing circuit (DSP), analog-to-digital converters (ADCs), and memory (e.g., RAM, Flash). The module executes the sophisticated algorithmic pipeline that transforms raw sensor data into a clinical diagnosis.

### Algorithmic Pipeline: From Signal to Diagnosis:

Step 1: Signal Acquisition and Pre-processing. The module digitizes the analog signals from each sensor in the array 200. This raw data then undergoes pre-processing, which includes noise reduction via digital filtering, baseline correction to compensate for sensor drift, and signal normalization to account for environmental variables.
Step 2: Feature Extraction. The algorithm extracts a vector of descriptive features from each sensor's time-series response curve. The raw data of changes detected by the sensor array is selected from the group consisting of the normalized change of sensor signal at the peak of the exposure, the normalized change of sensor signal at the middle of the exposure, the normalized change of sensor signal at the end of the exposure, and the area under the curve of the sensor signal, among others. The collection of features from all sensors forms a high-dimensional biosignature profile for the sample at a given point in time.
Step 3: Machine Learning Model Application. This biosignature profile is fed into a suite of pre-trained AI and machine learning algorithms. The algorithms may comprise one or more selected from the group consisting of: artificial neural networks (e.g., MLP, GRNN), support vector machines (SVM), principal component analysis (PCA), linear discriminant analysis (LDA), partial least squares (PLS), decision trees, and nearest neighbor analysis. These models perform the core diagnostic interpretation:
   i. Pathogen Presence Detection: A binary classification model analyzes the biosignature to determine if it is statistically distinct from a “healthy” or “sterile” baseline, yielding a positive/negative result for infection. The system's high sensitivity allows for detection of pathogen concentrations across a broad range, from as low as 1 to 108 CFU/mL of fluid or per gram of tissue.
   ii. Pathogen Identification: A multi-class classification or pattern recognition model compares the biosignature “fingerprint” to a comprehensive reference database containing patterns of known pathogens. This provides an assessment of the likely causative agent(s).
   iii. Microbial Load Estimation: A regression model establishes a correlation between the magnitude and shape of the biosignature and the microbial load, providing a semi-quantitative (e.g., Low, Medium, High) or quantitative (e.g., estimated CFU/mL) output.
   iv. Microbial Growth Rate Monitoring: This is a key inventive step for continuous monitoring. By acquiring biosignatures at regular intervals (e.g., every 0.1-60 minutes), the module creates a time-series of the estimated microbial load. It then calculates the rate of change (i.e., the first derivative) of this load over time. A positive rate indicates active proliferation, while a zero or negative rate can confirm the efficacy of antimicrobial treatment.

Furthermore, the AI analysis algorithms can be continually updated and improved based on ongoing data collection, research, and development, allowing for ever-increasing accuracy and the ability to detect newly emerging pathogens or resistance patterns.

### V. User Interface, Communication, and Data Management (400)

The reporting interface 400 ensures that the analytical results are delivered to the user in a clear, timely, and actionable manner.
A. On-Device Interface: The device may be equipped with a real-time display, such as an LCD screen, showing detected pathogen species, a risk scale, or a graph of bacterial load over time. Simpler embodiments may use colored LEDs for a quick status indication (e.g., Green/Yellow/Red). Visual or audible alarms can be configured to alert users to critical changes.
B. Wireless Communication and Telehealth Integration: The device includes wireless connectivity options such as Wi-Fi, Bluetooth (e.g., BLE), and Near-Field Communication (NFC). This facilitates seamless, secure data transmission to a companion mobile application on a smartphone or tablet, or directly to a central server. This enables remote monitoring by healthcare providers, supporting telehealth workflows.
C. Integration with Health Information Systems: The communication protocols are designed to allow for integration with hospital Electronic Health Record (EHR) systems. This allows infection data to be automatically logged into the patient's medical record, ensuring data integrity and facilitating better care coordination.

### VI. Exemplary Embodiments and Physical Configurations

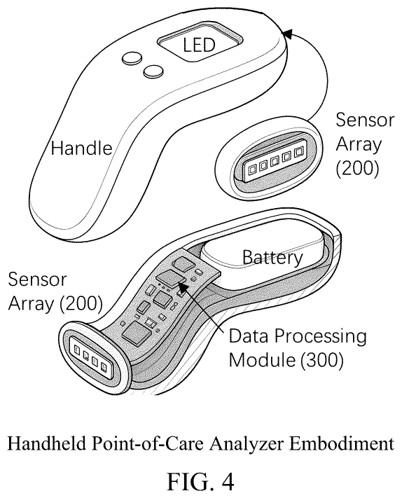

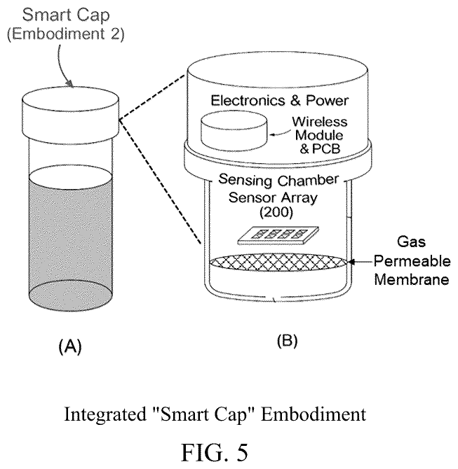

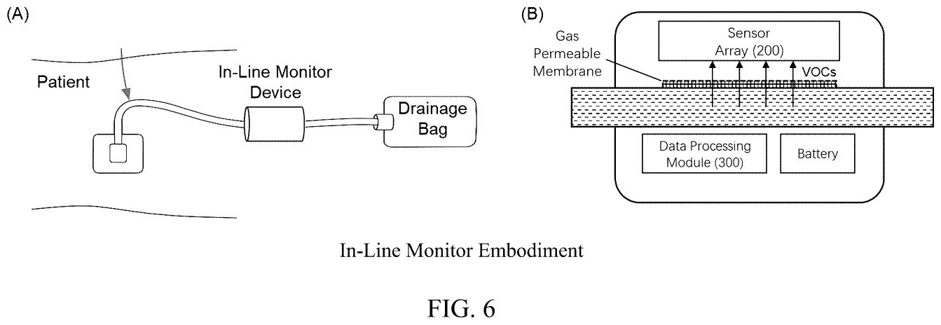

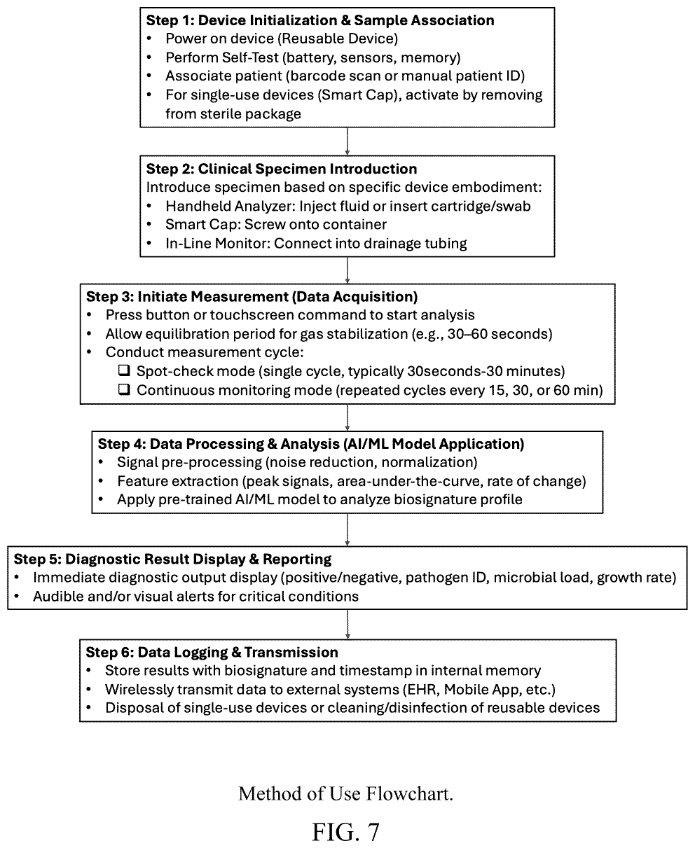

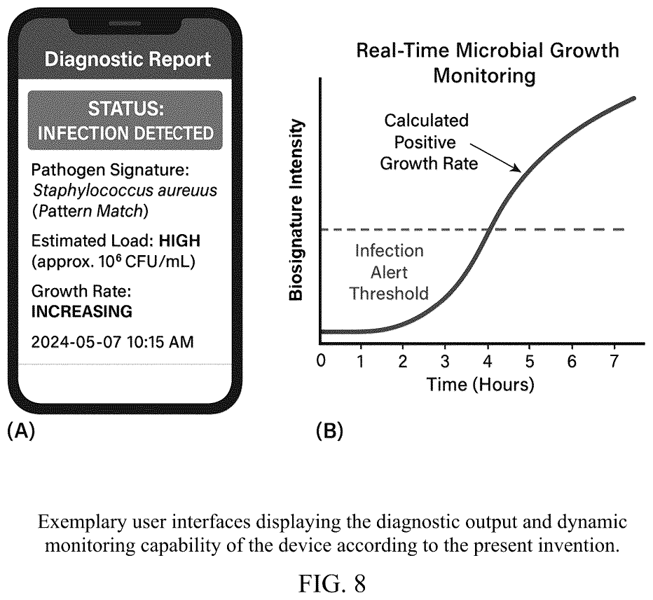

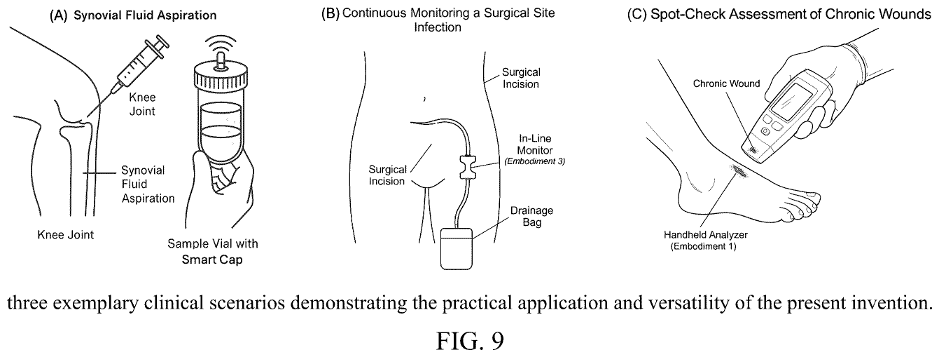

Referring now to FIG. 4-6, the device of the present invention can be realized in several distinct physical embodiments to suit diverse clinical needs and workflows. The following detailed descriptions of preferred embodiments are illustrative and not intended to be limiting.

### A. Embodiment 1: Handheld Point-of-Care Analyzer (FIG. 4)

In this embodiment, the device is configured as a reusable, ergonomic handheld analyzer, facilitating rapid, non-invasive diagnostic analysis at the point-of-care. As clearly depicted in FIG. 4, the device comprises a housing constructed from medical-grade polymer, such as Acrylonitrile Butadiene Styrene [ABS] or polycarbonate, ensuring durability, ease of sterilization, and suitability for clinical environments.
1. Sampling Interface: The analyzer integrates the sensor array (200) into a probe-like tip designed for direct, non-invasive interaction with the targeted skin area, such as a chronic wound (FIG. 9 panel (C)). This design eliminates internal sample chambers or fluidic pathways. Optionally, a single-use, disposable silicone contact ring may surround the sensor array to maintain consistent headspace volume during measurement, enhancing measurement reliability and minimizing cross-contamination risk.
2. Internal Components and Layout: As clearly depicted in FIG. 4, the handheld analyzer includes an internal data processing module (300) mounted on a printed circuit board, positioned proximally to the sensor array. This module processes sensor signals in real-time, facilitating immediate diagnostic results. Additionally, the device houses a rechargeable battery, strategically placed within the handle portion for balanced ergonomics.
3. User Interface and Controls: The analyzer features an integrated visual interface, such as an LED or OLED display, for instant readouts of diagnostic outcomes. Control buttons (e.g., power, start/stop) are ergonomically positioned for intuitive operation. Optionally, the device may incorporate wireless data transmission capabilities and an integrated barcode scanner, allowing rapid association with patient identifiers and seamless integration into clinical workflows.
4. Power and Operational Flexibility: The device's battery is rechargeable via a USB-C port. Additionally, alternative power options, such as conformal solar panels, may be integrated for applications in resource-limited environments, field hospitals, or remote clinical settings.
5. Through this compact, ergonomic, and versatile configuration, the handheld analyzer supports rapid clinical decision-making, improves patient care efficiency, and effectively integrates advanced diagnostic sensing capabilities into diverse healthcare scenarios.

### B. Embodiment 2: Integrated Smart Cap for Specimen Containers (FIG. 5)

This embodiment integrates the entire sensing and analysis system into a compact, disposable, sterile “Smart Cap,” designed explicitly for specimen containers such as urine cups or fluid vials. As shown in FIG. 5, this embodiment emphasizes simplified clinical workflow and reduced sample-handling risks.
1. Structural Design: The cap is constructed in two sealed compartments. The lower compartment, which faces the specimen, houses the miniaturized sensor array 200. It is protected from direct liquid contact by a microporous, hydrophobic membrane, which acts as a sterile barrier but allows volatile organic compounds (VOCs) and other gases to freely diffuse into the sensing chamber. The upper, fully-sealed compartment contains the data processing module 300, a coin-cell battery, and the wireless communication antenna, keeping all electronics isolated from the clinical sample.
2. Material and Activation: The cap is molded from a medical-grade polymer like polypropylene (PP) to ensure sterility and biocompatibility. To activate the device, a simple mechanism such as a pull-tab is used, which, when removed by the user, closes the battery circuit. Alternatively, the final twisting motion of securing the cap onto the container can trigger an internal micro-switch to power on the device.
3. Advantage: This “sample-in, result-out” design drastically simplifies the clinical workflow, requires minimal user training, and eliminates risks associated with sample transfer and handling.

### C. Embodiment 3: In-Line Monitor for Surgical Drains and NPWT Systems (FIG. 6)

The third embodiment provides continuous, non-invasive monitoring of post-operative drain fluids or wound exudates. FIG. 6 illustrates the in-line monitor's T- or Y-shaped transparent design for easy integration into standard medical tubing systems.
1. Physical Form and Connectivity: The module's housing is typically “T” or “Y” shaped and is molded from a transparent, medical-grade, biocompatible material such as silicone or PVC. It features standard-sized barbed or threaded connectors (e.g., to fit ¼ inch or 6 mm inner diameter tubing) at its ends, allowing for seamless, sterile insertion into existing medical tubing, such as between a surgical drain and its collection bulb or bag.
2. Sensing Principle: The module is designed with a separate sensing chamber that is adjacent to the main fluid path. A gas-permeable membrane separates the two, allowing headspace gases from the flowing fluid to diffuse into the sensing chamber and interact with the sensor array 200. This design ensures that the sensors continuously monitor the state of the fluid without ever coming into direct physical contact with it, thereby preventing biofouling and ensuring signal stability over long monitoring periods (e.g., 24-72 hours or 7 days).
3. Application: This embodiment is ideal for the early detection of Surgical Site Infections (SSIs). When installed in a patient's drain line post-surgery, it can detect the earliest metabolic signs of an impending infection long before systemic symptoms like fever or erythema appear.

### VII. Method of Use and Clinical Workflow

Referring now specifically to FIG. 7, a standardized, step-by-step clinical workflow for utilizing the invention is clearly illustrated. This workflow demonstrates the seamless integration of the device into existing medical practices:
Step 1: Preparation and Initialization.
   a. For a reusable device (e.g., Handheld Analyzer), the user powers on the device. The device performs a Power-On Self-Test (POST), checking battery status, sensor baseline stability, and memory.
   b. The user associates the test with a patient by either scanning a barcode on the patient's wristband or manually entering a unique patient identifier via the touchscreen.
   c. For a single-use device (e.g., Smart Cap), this step is simplified to remove the device from its sterile packaging and activating it (e.g., by pulling a tab).
Step 2: Sample Introduction.
   d. The user introduces the clinical specimen according to the specific embodiment. This could involve:
      i. Handheld: The operator hold the activated device to approach the point-of-interest for the test.
      ii. Smart Cap: Screwing the cap tightly onto the specimen container filled with urine or other fluid.
      iii. In-Line Monitor: This step is typically performed once, at the end of surgery, when the module is connected into the drainage tubing.
Step 3: Measurement and Analysis.
   e. The user initiates the measurement cycle via a button or touchscreen command. The system first allows for a brief “equilibration period” (e.g., 30-60 seconds) for the sample's headspace gas to stabilize.
   f. The system then enters the “measurement cycle”:
      i. In “Spot-Check Mode”, the cycle may last for 60 to 180 seconds, after which a final result is provided.
      ii. In “Continuous Monitoring Mode”, the device automatically repeats the measurement cycle at preset intervals (e.g., every 15, 30, or 60 minutes) to track changes over time and calculate the microbial growth rate.
Step 4: Result Interpretation and Clinical Decision.
   g. Upon completion of the analysis, the diagnostic output is immediately displayed on the user interface 400.
   h. A clear, actionable result (e.g., “Positive: Pseudomonas aeruginosa pattern detected, Estimated Load: High, Growth Rate: Increasing”) empowers the clinician to make rapid decisions, such as initiating or modifying antibiotic therapy, ordering surgical debridement, or obtaining further imaging studies.
Step 5: Data Logging and Post-Analysis Actions.
   i. The test result, including the full biosignature profile and a timestamp, is automatically saved to the device's internal memory and, if connected, is wirelessly transmitted to the patient's Electronic Health Record (EHR) for documentation and future analysis.
   j. Single-use devices are disposed of according to standard medical waste protocols. Reusable devices may undergo a cleaning and disinfection cycle as per the manufacturer's instructions.

### VIII. User Interface and Dynamic Monitoring Output

Referring now specifically to FIG. 8, representative user interfaces demonstrating the diagnostic output and dynamic monitoring capabilities of the present invention are clearly illustrated. The user interfaces exemplify how diagnostic results and microbial trends are effectively communicated to clinicians in real-time, ensuring timely and informed clinical decision-making.
Part (A): Diagnostic Result Display: Depicted here is a typical handheld analyzer or mobile application interface clearly providing immediate diagnostic feedback. The screen prominently displays essential diagnostic information such as pathogen identification (e.g., “Positive: Pseudomonas aeruginosa detected”), estimated microbial load (“Microbial Load: HIGH”), timestamp of measurement, patient identification details, and intuitive visual or audible alerts to promptly notify clinical staff of critical findings.
Part (B): Dynamic Monitoring Curve: This graphical interface showcases continuous monitoring data represented as a clear, intuitive graph plotting microbial load or biosignature signal intensity against time. The trend line distinctly illustrates microbial growth rates, facilitating predictive monitoring and enabling clinicians to visually track infection progression or response to treatment over multiple measurement intervals. Critical thresholds for microbial load or significant rate changes may trigger automatic alerts, prompting immediate clinical intervention or further investigation.

The clearly structured user interfaces illustrated in FIG. 8 highlight the invention's practical utility in clinical environments by providing actionable diagnostic insights and real-time infection monitoring capability. This ensures clinicians can rapidly interpret, document, and act upon critical diagnostic data, greatly enhancing clinical workflow efficiency, patient management, and therapeutic outcomes.

### IXV. Exemplary Clinical Use Cases

Referring now specifically to FIG. 9, three representative clinical scenarios are visually illustrated, clearly demonstrating the practical utility and clinical value of the present invention across diverse medical applications.

### A. Use Case 1: Rapid Diagnosis of Prosthetic Joint Infection (PJI)

1. Clinical Scenario: A patient who underwent a total knee arthroplasty one year prior presents with persistent knee pain and swelling. The clinician suspects a low-grade PJI, a condition that is notoriously difficult to diagnose with traditional, slow, and often insensitive serological markers.
2. Workflow: The clinician aspirates the patient's knee joint under sterile conditions, drawing approximately 0.5 mL of synovial fluid into a syringe (FIG. 9 panel (A)). The fluid is immediately injected into a sample vial sealed with the integrated Smart Cap (Embodiment 2) for rapid analysis.
3. Clinical Value: Within minutes, while the patient is still in the examination room, the Smart Cap's interface displays a “Positive” result, indicating that the biosignature pattern is highly consistent with that of Staphylococcus epidermidis at a moderate estimated load. This immediate, point-of-care result provides powerful evidence to support the decision for surgical intervention (debridement and implant retention), bypassing the typical 2-5 day wait for microbiology culture results and dramatically improving the chances of a successful outcome.

### B. Use Case 2: Continuous Monitoring for Post-Operative Surgical Site Infection (SSI)

1. Clinical Scenario: Following major abdominal surgery (e.g., a colectomy), a patient receives a surgical drainage tube to manage postoperative fluids, posing significant infection risks.
2. Workflow: At surgery completion, the clinician integrates the In-Line Monitor (Embodiment 3) directly into the patient's surgical drainage tubing connected to a collection bag (FIG. 9 panel (B)). The monitor autonomously measures and analyzes headspace gases emitted from drain fluid every 30 minutes, continuously tracking potential infection indicators.
3. Clinical Value: On post-operative day 2, at 3:00 AM, the system detects a sharp, exponential increase in the calculated microbial growth rate. The system automatically sends a “High Risk” alert to the central nursing station monitor and pushes a notification to the on-call surgeon's smartphone. This alert occurs a full 24 hours before the patient develops a fever or other clinical signs of infection. Acting on this early warning, the medical team orders an urgent CT scan, which identifies a small, developing intra-abdominal fluid collection. They are able to initiate targeted antibiotic therapy immediately, preventing the progression to a life-threatening abscess and peritonitis.

### C. Use Case 3: Bioburden Management in Chronic Wounds (e.g., Diabetic Foot Ulcers)

1. Clinical Scenario: A patient with diabetes attends a weekly wound care clinic for the management of a chronic foot ulcer. A key challenge is distinguishing between benign bacterial colonization and a critical level of bioburden (infection) that is impairing healing.
2. Workflow: At the point of care, the clinician directly positions the Handheld Analyzer (Embodiment 1) over the chronic wound site, performing a rapid, non-invasive spot-check analysis (FIG. 9 panel (C)). The analyzer directly samples gaseous emissions from the wound without physical contact, analyzing biosignatures immediately.
3. Clinical Value: Within 2 minutes, the analyzer's screen displays the wound's status as “High-Critical Colonization” and identifies the dominant pathogen signature as consistent with Staphylococcus aureus (MRSA). This immediate, objective data allows the clinician to perform a more aggressive debridement on the spot and to select an appropriate topical antimicrobial dressing known to be effective against MRSA. This replaces the old, inefficient workflow of “swab, send to lab, wait 3 days for results, and adjust treatment at the next visit,” enabling precise, data-driven wound care in a single visit.

While the present disclosure has been described with reference to specific embodiments, it will be understood by those skilled in the art that various changes may be made, and equivalents may be substituted without departing from the scope of the disclosure. In addition, many modifications may be made to adapt a particular situation or material to the teachings of the disclosure without departing from the essential scope thereof. Therefore, it is intended that the present disclosure not be limited to the particular embodiments disclosed, but that the disclosure will include all embodiments falling within the scope of the appended claims.

We claim:

1. A device for analyzing a clinical specimen, the device comprising:
a) a sensor array configured to interface with gaseous analytes released from the clinical specimen, said sensor array comprising at least one chemiresistive gas sensor;
b) a data processing module communicatively coupled to said sensor array, the data processing module configured to: i. receive a plurality of sensor signals from said sensor array over a period of time or during a short-duration cycle to generate a biosignature profile, wherein the profile may be time-resolved or derived from a spot-check measurement; and ii. analyze said time-resolved biosignature profile to determine at least one characteristic of the specimen selected from the group consisting of: pathogen presence, pathogen identity, an estimation of microbial load, and a determination of microbial growth rate; wherein said determination of microbial growth rate is calculated from a rate of change of said time-resolved biosignature profile over said period of time; and
c) a user interface for reporting said at least one characteristic.

2. The device of claim 1, wherein said sensor array further comprises at least one auxiliary sensor selected from the group consisting of: a pH sensor, a temperature sensor, an optical sensor, a humidity sensor, and an impedance/conductance sensor.

3. The device of claim 1, wherein said estimation of microbial load is correlated to a predicted pathogen concentration expressed in Colony Forming Units (CFU).

4. The device of claim 1, wherein said data processing module utilizes one or more machine learning models to analyze said time-resolved biosignature profile.

5. The device of claim 4, wherein said one or more machine learning models are selected from the group consisting of: a support vector machine (SVM), an artificial neural network, a deep neural network (DNN), a convolutional neural network (CNN), a recurrent neural network (RNN), a gradient boosting machine (e.g., XGBoost), a random forest, a k-nearest neighbors (k-NN) algorithm, or a partial least squares (PLS) regression.

6. The device of claim 1, wherein said at least one chemiresistive gas sensor comprises a sensing material selected from the group consisting of: a metal oxide semiconductor, a carbon-based nanomaterial, a conductive polymer, and composites thereof.

7. The device of claim 6, wherein said metal oxide semiconductor comprises a material composed of a unary, binary, ternary, quaternary, quinary, senary, septenary, or octonary multiple-component metal oxide.

8. The device of claim 1, wherein the device is configured as a handheld point-of-care analyzer.

9. The device of claim 1, wherein the device is configured as a cap for a specimen container.

10. The device of claim 1, wherein the device is configured as an in-line monitoring module for a surgical drainage system or a negative pressure wound therapy system.

11. The device of claim 1, wherein said user interface comprises a wireless communication module for transmitting said at least one characteristic to an external device.

12. The device of claim 1, wherein said clinical specimen is selected from the group consisting of: a body fluid and a tissue sample.

13. A method for analyzing a clinical specimen, the method comprising the steps of:
a) exposing a sensor array comprising at least one chemiresistive gas sensor to gaseous analytes released from the clinical specimen;
b) acquiring, via a data processing module over a period of time, a plurality of sensor signals from said sensor array to generate a time-resolved biosignature profile;
c) analyzing, via the data processing module, said time-resolved biosignature profile to determine at least one characteristic of the specimen selected from the group consisting of: pathogen presence, pathogen identity, an estimation of microbial load, and a determination of microbial growth rate;
wherein the step of determining the microbial growth rate comprises calculating a rate of change of said time-resolved biosignature profile over said period of time; and
d) reporting, via a user interface, said at least one characteristic.

14. The method of claim 13, wherein the analyzing step is performed utilizing one or more machine learning models.

15. The method of claim 13, wherein the step of determining the estimation of microbial load comprises correlating features of the biosignature profile to a predicted pathogen concentration expressed in Colony Forming Units (CFU).

16. The method of claim 13, wherein the exposing step is performed over a continuous duration to provide continuous monitoring of the clinical specimen.

17. The method of claim 16, wherein the sensor array is integrated into an in-line module connected within a surgical drainage line.

18. The method of claim 13, wherein the exposing step is performed over a short duration to provide a spot-check analysis of the clinical specimen.

19. The method of claim 18, wherein the sensor array is integrated into a cap of a specimen container.

20. The method of claim 13, further comprising the step of transmitting, via a wireless communication module, said at least one characteristic to an external electronic health record system.
